(19) United States
(12) Patent Application Publication
(10) Pub. No.: US 2022/0218647 A1
(43) Pub. Date: Jul. 14, 2022

(54) DIETARY BUTYRATE

(71) Applicant: SOCIETE DES PRODUITS NESTLE S.A., Vevey (CH)

(72) Inventors: LUCAS ACTIS GORETTA, Singapore (SG); MOHAMED NABIL BOSCO, Epalinges (CH); AMAURY PATIN, St. Louis, MO (US)

(21) Appl. No.: 17/595,581

(22) PCT Filed: May 20, 2020
(86) PCT No.: PCT/EP2020/064068

§ 371 Date: Nov. 19, 2021

(30) Foreign Application Priority Data
May 21, 2019 (EP) 19175553.7

(51) Int. Cl. A61K 31/231

(52) U.S. Cl. CPC A61K 31/231

(57) ABSTRACT

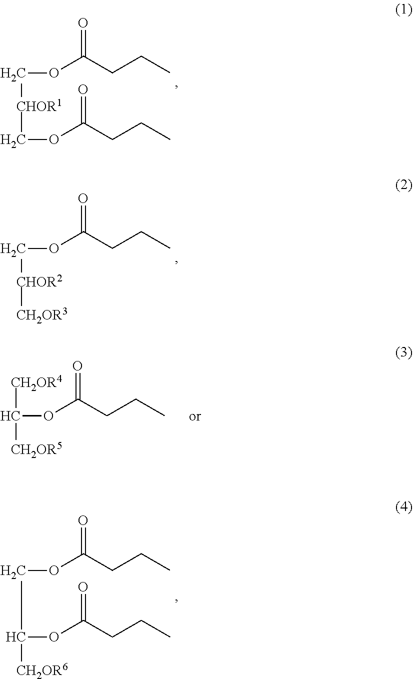

A compound having the formula (1), (2), (3) or (4), or combinations thereof, for use in the treatment or prevention of cardiovascular disorders in a subject, wherein R¹, R², R³, R⁴, R⁵ and R⁶ are independently, a long chain fatty acid having between 16 and 20 carbons.

### FIELD OF THE INVENTION

The present invention relates to a dietary source of butyrate having improved organoleptic properties for use in the prevention and/or treatment of cardiovascular disorders.

### BACKGROUND TO THE INVENTION

Cardiovascular disorders account for approximately 20% of all annual worldwide deaths, and remain the leading cause of death in both developed and developing countries (S. K. Wattanapitayakul et al., Pharmacology and Therapeutics, 89, 187-206 (2001)). Cardiovascular disorders are also a leading cause of disability in later life. Cardiovascular disorders affect the heart or blood vessels. A major cardiovascular disorder is atherosclerosis (also known as arteriosclerotic vascular disease) which is a condition in which patchy deposits of fatty material (atheromas or atherosclerotic plaques) develop in the walls of medium-sized and large arteries, leading to reduced or blocked blood flow. Atherosclerosis is one form of arteriosclerosis, which means hardening of the arteries. Arteriosclerosis interferes with the body's control of blood pressure, increasing the risk of high blood pressure. The stiffness of the arteries prevents the dilation that would otherwise return blood pressure to normal. People with high blood pressure are at a greater risk of stroke, heart attack, and kidney failure.

The main cause of atherosclerosis is yet unknown, it is a syndrome affecting arterial blood vessels, a chronic inflammatory response in the walls of arteries, caused largely by the accumulation of macrophages and promoted by low-density lipoproteins (LDL, plasma lipoproteins that carry cholesterol and triglycerides) without adequate removal of fats and cholesterol from the macrophages by functional high density lipoproteins (HDL). It is commonly referred to as a hardening or furring of the arteries. This is caused by the formation of multiple plaques within the arteries.

Where atherosclerosis affects the arteries supplying blood to the heart (coronary artery disease) this can cause chest pain (angina pectoris) or a heart attack where an area of heart muscle is destroyed (myocardial infarction). The reduction of the flow of oxygen-rich blood to the heart muscle can cause heart failure, a disorder in which the heart pumps blood inadequately, leading to reduced blood flow, back-up (congestion) of blood in the veins and lungs, and other changes that may further weaken the heart. The failure of coronary circulation to supply adequate circulation to cardiac muscle and surrounding tissue is called coronary heart disease.

Atherosclerosis that affects the arteries to the brain leads to strokes. A stroke occurs when an artery to the brain becomes blocked or ruptures, resulting in death of an area of brain tissue (cerebral infarction).

Although the major cardiovascular disorders in terms of mortality are strokes and heart attacks, cardiovascular disorders also encompass such conditions as aortic aneurysms and peripheral vascular disease and contribute to clinical conditions including renal vascular disease, vascular dementia and retinal disease. Preventing cardiovascular disorders is not just about reducing mortality, but also about preventing disability and improving quality of life.

Initial treatment for cardiovascular disorders focuses on diet and lifestyle interventions, for example increasing exercise, eating a low fat diet and stopping smoking.

A large number of compounds have been proposed for the treatment or prevention of cardiovascular disorders. These include: salicylates (WO2009/006583), statins (J. K. Liao, International Journal of Cardiology, 86, 5-18 (2002)), angiotensin-converting enzyme (ACE) inhibitors (U.S. Pat. No. 5,684,016), angiotensin II receptor blockers (EP1604664) and calcium channel blockers (EP0089167).

Unfortunately, the treatments currently available are not always entirely satisfactory, in particular in terms of efficacy and/or in terms of the side effects which may be associated with them.

It is known that many patients under statins remain at increased risk of CVD. Those patients have persistent elevations in hsCRP despite significant reductions in LDL-C levels. This defined a so-called “residual inflammatory risk” and has become a pharmacologic target of interest. Evidence of the relevance of this target came from the Canakinumab Anti-inflammatory Thrombosis Outcome Study (CANTOS) trial. Here it was shown that direct reduction of inflammation with an IL-1β antagonist reduces cardiovascular event rates independently of LDL-C values (P. M. Ridker, N Engl J Med. 2017 Sep 21;377(12):1119-1131).

However, IL-1β antagonist treatment has a prohibitive cost and important side effects.

It would thus be highly desirable to have additional compositions available for the treatment or prevention of cardiovascular disorders without the drawbacks of those described in the prior art.

In human large intestine acetate, propionate, and butyrate are the predominant short chain fatty acids generated by microbiota after fermentation of resistant starch or dietary fiber. Once produced, SCFAs are absorbed by enterocytes of the intestines as an energy source, but a portion of these absorbed fatty acids reaches the systemic circulation and modulates the biological activities of multiple biological systems and or organs. There is evidence that SCFAs are able to have anti-inflammatory effects and attenuate the development of a variety of inflammatory diseases including inflammatory bowel disease, colon cancer and diabetes mellitus (P. A. Gill, Aliment Pharmacol Ther. 2018 Jul;48(1):15-34).

However, the effects of SCFAs on atherosclerosis and subsequent cardiovascular diseases remain poorly understood. Arthrosclerosis is a general term describing any hardening (and loss of elasticity) of medium or large arteries due to an atheromatous plaque formation into the arteries. Vascular researchers have documented role of inflammation in atherosclerosis earlier. During atherosclerotic plaque initiation, macrophages release proinflammatory cytokines that activate endothelial cells and recruit additional leukocytes. This causes adhesion of leukocytes to endothelial cells, transmigration of leukocytes to the subendothelial space, and plaque progression and maturation. Inside plaques, inflammatory signals from activated leukocytes (particularly IL1 and I L6) will ultimately trigger plaque destabilization and rupture, as seen in myocardial infraction (P. M. Ridker, Circ Res. 2016 Jan 8;118(1):145-56, P. M. Ridker, Circ Res. 2019 Feb; 124(3):437-450).

Recent clinical trial data have now demonstrated that inflammation is a key driver of atherosclerosis as suggested earlier with epidemiological data. Circulating biomarkers of inflammation, including high-sensitivity C-reactive protein (hsCRP) and interleukin-6 (IL-6), are associated with increased risk of cardiovascular events. This is independent of cholesterol and other traditional risk factors. Indeed, randomized trials have shown that statin treatment also reduces hsCRP in addition to its blood lipid-lowering effect (low-density lipoprotein cholesterol aka LDL-C). Interestingly, the magnitude of hsCRP reduction is proportional to the reduction in cardiovascular risk (P. M. Ridker, J Am Coll Cardiol. 2016 Feb 16;67(6):712-723).

In-vitro studies have demonstrated that, after challenge, endothelial inflammatory response is also reduced by SCFA pre-treatment. LPS or TNF challenged endothelial cells pre-treated with SCFAs secrete less interleukin-6, express less Vascular cell adhesion protein 1 (VCAM1), and reduce leukocyte adhesion. (M. Li, Front Pharmacol. 2018 May 23;9:533).

In other studies, in cultured endothelial cells, butyrate has been found to significantly decrease the formation and activation of Nrlp3 inflammasome induced by cholesterol crystals, while acetate or propionate did not. Further in vivo investigations demonstrated that butyrate has protective effects on arterial wall in a rodent model of carotid artery injury associated to poor Nrlp3 inflammasome complex formation and activation. (X. Yuan, Redox Biol. 2018 Jun;16:21-31).

These in-vitro and in-vivo studies support the anti-inflammatory role of SCFA and make butyrate a candidate for prevention and/or treatment of atherosclerosis.

Common sources of butyrate are butyric acid and tributyrin, a triglyceride made of three ester functional groups with three butyrate moieties and the glycerol backbone. Butyric acid and tributyrin are both food additives that are generally regarded as safe (GRAS) (21CFR582.60 and 21CFR184.1903 respectively), and are natural components of many dairy items. However, butyric acid is associated with negative sensory qualities such as vomit-like, fecal, and cheesy aroma attributes. Tributyrin also has negative sensory qualities, in particular high bitterness. These unpleasant taste and odor attributes can make the oral administration of compositions including these compounds particularly difficult. Butyrate components from dairy cannot be enriched and thus significant volumes of dairy fat would need to be consumed which is not feasible for practical and nutritional reasons, not least as it would lead to large amount of unwanted calorie derived from animal fat.

While butyrate could be beneficial for prevention and/or treatment of atherosclerosis, today, there is no method to deliver butyrate in a form that has acceptable organoleptic properties.

Accordingly, it would be beneficial to provide a food-grade source of butyrate having improved organoleptic properties as compared to available solutions for use in the treatment or prevention of cardiovascular disorders.

### SUMMARY OF THE INVENTION

The present invention provides compounds that are a source of butyrate having improved organoleptic properties for use in preventing or treating cardiovascular diseases, The compounds have improved odor and/or taste relative to butyric acid, butyrate salts and/or tributyrin. The compounds may be used as a dietary source of butyric acid. The compounds may be used in, for example, nutritional compositions, dietary supplements, beverages and pet care products.

The compounds and compositions may be used in the prevention or treatment of cardiovascular disorders, in particular atherosclerosis, through reduction of residual inflammatory risk. The cardiovascular disorders may be selected from the group consisting of atherosclerosis, coronary heart disease, myocardial infarction, angina pectoris, stroke, and/or heart failure. As discussed above, stroke and myocardial infarction (heart attack) are the major cardiovascular disorders in terms of mortality. Coronary heart disease and heart failure are also significant causes of death and debilitating ill health. Atherosclerosis is the underlying cause of many other cardiovascular disorders. Many people live with high blood pressure and regular bouts of angina pectoris, but quality of life is often affected and the people have a constant concern that these may be the prelude to strokes, heart failure or heart attacks. It would therefore be advantageous to treat or prevent atherosclerosis, coronary heart disease, myocardial infarction, angina pectoris, stroke, and/or heart failure.

According to a first aspect of the present invention there is provided a compound having the formula

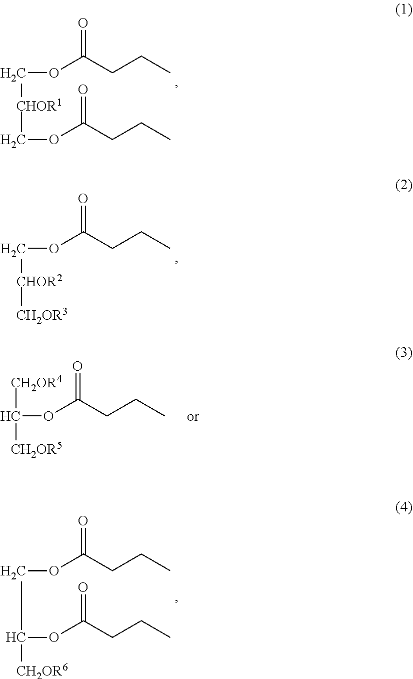

or combinations thereof, for use in the prevention or treatment of cardiovascular disorders in a subject, wherein R¹, R², R³, R⁴, R⁵ and R⁶ are independently, a long chain fatty acid having between 16 and 20 carbons.

According to another aspect of the present invention there is provided a composition comprising a compound having the formula (1), (2), (3) or (4) or combinations thereof, for use in the prevention or treatment of cardiovascular disorders in a subject, wherein R¹, R², R³, R⁴, R⁵ and R⁶ are independently, a long chain fatty acid having between 16 and 20 carbons.

In one embodiment, the composition comprises the compound having formula (1), the compound having formula (2), the compound having formula (3) and the compound having formula (4).

The composition may comprise the compound having formula (1) and the compound having formula (2).

The composition may comprise the compound having formula (1) and the compound having formula (3).

The composition may comprise the compound having formula (1) and the compound having formula (4).

The composition may comprise the compound having formula (2) and the compound having formula (3).

The composition may comprise the compound having formula (2) and the compound having formula (4).

The composition may comprise the compound having formula (3) and the compound having formula (4).

The composition may comprise the compound having formula (1) the compound having formula (2), and the compound having formula (3).

The composition may comprise the compound having formula (1) the compound having formula (2), and the compound having formula (4).

The composition may comprise the compound having formula (1) the compound having formula (3), and the compound having formula (4).

The composition may comprise the compound having formula (2) the compound having formula (3), and the compound having formula (4).

The composition may comprise the compound having formula (1), the compound having formula (2), the compound having formula (3) and the compound having formula (4).

In one embodiment the compounds having formula (1), (2), (3) and (4), comprise at least 50%, 60%, 70%, 80%, 90%, 95% or 99% by weight of the total triglycerides of the composition.

In one embodiment the compounds having formula (1), (2), (3) and (4), comprise at least 50%, 60%, 70%, 80%, 90%, 95% or 99% by weight of the total butyrate moiety containing triglycerides in the composition.

In one embodiment tributyrin comprises less than 10% by weight of the total triglycerides in the composition, preferably less than 8% by weight, more preferably less than 5% by weight of the total triglycerides in the composition.

In one embodiment the composition further comprises dietary fiber and/or probiotic.

The composition of the invention may be in the form of a nutritional composition, for instance a food, a beverage, a pet care product.

The composition of the invention may be in the form of a dietary supplement.

In one embodiment R¹, R², R³, R⁴, R⁵ and/or R⁶ is an unsaturated fatty acid, preferably monounsaturated.

In one embodiment R¹, R², R³, R⁴, R⁵ and/or R⁶ is selected from the group consisting of oleic acid, palmitic acid, or linoleic acid, preferably each of R¹, R², R³, R⁴, R⁵ and R⁶ is oleic acid.

The compounds and compositions of the present invention may be useful for preventing or treating cardiovascular disorders.

The compounds and compositions of the present invention, may be useful for preventing initiation and or progression of cardiovascular disorders, in particular atherosclerosis, and atherosclerosis associated disorders, by reduction of residual inflammatory risk.

In one embodiment the compounds or combinations thereof have improved organoleptic properties relative to butyric acid, tributyrin and/or butyrate salts.

In one embodiment the compounds is provided to a mammal, preferably, a human, a pet or a farm animal.

### BRIEF DESCRIPTION OF THE DRAWINGS

FIG. 1 shows the release of fatty acid from emulsions containing 200 mg of (A) tributyrin, (B) high oleic sunflower oil and (C) a mixture of butyrate moiety containing triacylglycerol (TAG) according to the invention, digested either with i) simulated intestinal fluid (SIF) or (ii) sequentially with gastric fluid (SGF) followed by simulated intestinal fluid (SIF).

FIG. 2 shows the overall extent of lipid digestion after both SIF and SGF-SIF for tributyrin, high oleic sunflower oil and a mixture of butyrate moiety containing TAG according to the invention.

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

### Triglycerides

A triglyceride (also known as a triacylglycerol) is a triester that is derived from glycerol and three fatty acids. Under hydrolysis conditions such as those during digestion, triglycerides may be a source of fatty acids. For instance, tributyrin is potentially a source of three moles of butyric acid per mole of tributyrin.

Fatty acids are carboxylic acids with a long tail (chain). Fatty acids may be either unsaturated or saturated. Fatty acids which are not attached to other molecules are referred to as free fatty acids (FFA).

The term “fatty acid moiety” refers to the part of the triglyceride that originates from a fatty acid in an esterification reaction with glycerol. The triglycerides used in the present invention comprise at least one butyric acid moiety and at least one long chain fatty acid moiety.

Preferred long chain fatty acids for use in the present invention are fatty acids that have 16 to 20 carbon atoms. Examples of long chain fatty acid include oleic acid, palmitic acid, stearic acid and linoleic acid. Preferably, the long chain fatty acid is oleic acid. For example, the present invention provides a compound having the formula

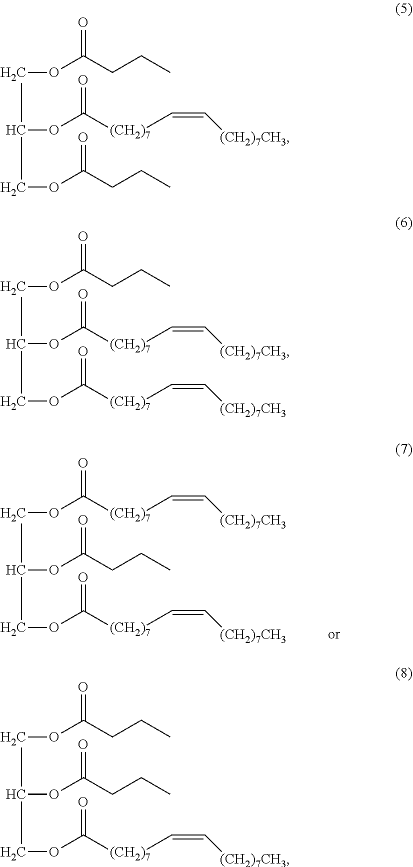

or combinations thereof, for use in the treatment or prevention of cardiovascular disorders subject.

Other examples of triglycerides which may be used in the present invention include: 1,3-dibutyryl-2-linoleoylglycerol, 1,3-dibutyryl-2-stearoylglycerol, 1-butyryl-2-oleoyl-3-palmitoylglycerol, 1-palm itoyl-2-oleoyl-3-butyrylglycerol, 1-butyryl-2-oleoyl-3-linoleoylglycerol, 1-linoleoyl-2-oleoyl-3-butyrylglycerol, 1-oleoyl-2-butyryl-3-linoleoylglycerol, 1-linoleoyl-2-butyryl-3-oleoylglycerol, 1-butyryl-2-linoleoyl-3-oleoylglycerol, 1-oleoyl-2-linoleoyl-3-butyrylglycerol, 1-butyryl-2-stearoyl-3-oleoylglycerol, 1-oleoyl-2-stearoyl-3-butyrylglycerol, 1-butyryl-2-oleoyl-3-stearoylglycerol, 1-stearoyl-2-oleoyl-3-butyrylglycerol, 1,2-dioleoyl-3-palmitoylglycerol, 1-palmitoyl-2,3-dioleoylglycerol, 1,2-dioleoyl-3-linoleoylglycerol and 1-linoleoyl-2,3-dioleoylglycerol.

The triglycerides of the present invention may be synthesised by, for example, esterification of long chain fatty acid(s) and butyric acid with glycerol.

The triglycerides of the present invention may be synthesised by, for example, interesterification between tributyrin and another triglyceride containing long chain fatty acids. In one embodiment, high oleic sunflower oil is the source of the long chain fatty acids. This generates triglycerides containing predominantly butyrate and oleate moieties. The compounds are dairy-free, cholesterol-free and vegan. Fatty acids are liberated from triglycerides due to lipases, naturally present in the gastrointestinal tract. Relative to butyrate salts, the compounds do not add additional mineral salts to the final formulation.

Alternative methods of triglyceride synthesis can be routinely determined by a person skilled in the art. By way of example, a method of obtaining 1,3-dibutyryl-2-palmitoylglycerol (BPB) is shown below:

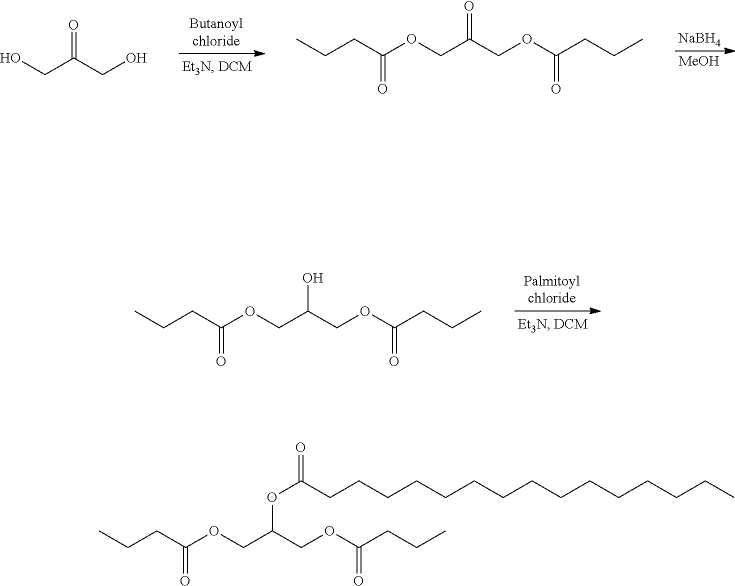 [SMILES: "MeOH" ; O=C(CO)CO ; CCCC(=O)OCC(=O)COC(=O)CCC ; "NaBH4" ; CCCC(=O)OCC(O)COC(=O)CCC ; CCCCCCCCCCCCCCCC(=O)OC(COC(=O)CCC)COC(=O)CCC]

A single butyrate moiety containing triglyceride may be used herein. Alternatively, a mixture of different butyrate moiety containing triglycerides may be used.

### Compositions

Compounds of the present invention may be administered in the form of a composition. Thus, the present invention provides compositions comprising butyrate moiety containing triglycerides referred to herein, for use in the prevention or treatment of cardiovascular disorders in a subject.

In one embodiment, a combination of a compound having formula (1) and a compound having formula (2) is present in the composition as defined herein.

In one embodiment the compound having formula (1) is present in an amount of at least 10% by weight of the total triglycerides in the composition, and the compound having formula (2) is present in an amount of at least 10% by weight of the total triglycerides in the composition.

In one embodiment the compound having formula (1) is present in an amount of at least 15% by weight of the total triglycerides in the composition, and the compound having formula (2) is present in an amount of at least 15% by weight of the total triglycerides in the composition.

In one embodiment the compound having formula (1) is present in an amount of at least 20% by weight of the total triglycerides in the composition, and the compound having formula (2) is present in an amount of at least 20% by weight of the total triglycerides in the composition.

In one embodiment the compound having formula (1) is present in an amount of at least 20% by weight of the total triglycerides in the composition, and the compound having formula (2) is present in an amount of at least 30% by weight of the total triglycerides in the composition.

In one embodiment the compound having formula (1) comprises about 20% to about 40% by weight of the total triglycerides in the composition, and/or the compound having formula (2) comprises about 30% to about 40% by weight of the total triglycerides in the composition.

In one embodiment the compound having formula (1) and the compound having formula (2) comprise at least 20%, 30%, 40%, 50%, 60% or 70% by weight of the total triglycerides in the composition, preferably about 40% to about 80%, or about 50% to about 75% by weight of the total triglycerides in the composition.

In one embodiment the composition further comprises the compound having formula (3), preferably wherein the compound having formula (3) comprises at least 2%, 3%, 4% or 5% by weight of the total triglycerides in the composition, and/or the composition further comprises the compound having formula (4), preferably wherein the compound having formula (4) comprises at least 1%, 2% or 3% by weight of the total triglycerides in the composition.

In one embodiment the compound having formula (1) is present in an amount of at least 20% by weight of the total butyric acid containing triglycerides in the composition, and the compound having formula (2) is present in an amount of at least 30% by weight of the total butyric acid containing triglycerides in the composition.

In one embodiment the compound having formula (1) comprises about 30% to about 50% by weight of the total butyric acid containing triglycerides in the composition, and/or the compound having formula (2) comprises about 40% to about 60% by weight of the total butyric acid containing triglycerides in the composition.

In one embodiment the compound having formula (1) and the compound having formula (2) comprise at least 20%, 30%, 40%, 50%, 60%, 70% or 80% by weight of the total butyric acid containing triglycerides in the composition, preferably about 60% to about 90% by weight of the total butyric acid containing triglycerides in the composition.

In one embodiment the compound having formula (5) comprises at least 10% by weight of the total triglycerides in the composition, and/or the compound having formula (6) comprises at least 10% by weight of the total triglycerides in the composition.

In one embodiment the compound having formula (5) comprises at least 15% by weight of the total triglycerides in the composition, and/or the compound having formula (6) comprises at least 15% by weight of the total triglycerides in the composition.

In one embodiment the compound having formula (5) comprises at least 15% by weight of the total triglycerides in the composition, and/or the compound having formula (6) comprises at least 20% by weight of the total triglycerides in the composition.

In one embodiment the compound having formula (5) comprises at least 20% by weight of the total triglycerides in the composition, and/or the compound having formula (6) comprises at least 20% by weight of the total triglycerides in the composition.

In one embodiment the compound having formula (5) comprises about 15% to about 30% by weight of the total triglycerides in the composition, and/or the compound having formula (6) comprises about 20% to about 30% by weight of the total triglycerides in the composition.

In one embodiment the compound having formula (5) and the compound having formula (6) comprise at least 20%, 30% or 40% by weight of the total triglycerides in the composition, preferably about 30% to about 60%, or about 40% to about 50% by weight of the total triglycerides in the composition.

In one embodiment the composition further comprises the compound having formula (7), preferably wherein the compound having formula (7) comprises at least 2% or 3% by weight of the total triglycerides in the composition, and/or the composition further comprises the compound having formula (8), preferably wherein the compound having formula (8) comprises at least 2% or 3% by weight of the total triglycerides in the composition.

In one embodiment the compound having formula (5) comprises at least 10% by weight of the total butyrate moiety containing triglycerides in the composition, and the compound having formula (6) comprises at least 10% by weight of the total butyrate moiety containing triglycerides in the composition.

In one embodiment the compound having formula (5) comprises at least 15% by weight of the total butyrate moiety containing triglycerides in the composition, and the compound having formula (6) comprises at least 15% by weight of the total butyrate moiety containing triglycerides in the composition.

In one embodiment the compound having formula (5) comprises at least 15% by weight of the total butyrate moiety containing triglycerides in the composition, and the compound having formula (6) comprises at least 20% by weight of the total butyrate moiety containing triglycerides in the composition.

In one embodiment the compound having formula (5) comprises at least 20% by weight of the total butyrate moiety containing triglycerides in the composition, and the compound having formula (6) comprises at least 20% by weight of the total butyrate moiety containing triglycerides in the composition.

In one embodiment the compound having formula (5) comprises about 15% to about 30% by weight of the total butyrate moiety containing triglycerides in the composition, and the compound having formula (6) comprises about 20% to about 30% by weight of the total butyrate moiety containing triglycerides in the composition.

In one embodiment the composition further comprises the compound having formula (7), preferably wherein the compound having formula (7) comprises at least 2% or 3% by weight of the total butyrate moiety containing triglycerides in the composition, and/or the composition further comprises the compound having formula (8), preferably wherein the compound having formula (8) comprises at least 2% or 3% by weight of the total butyrate moiety containing triglycerides in the composition.

In one embodiment composition of the present invention may further comprise 1,3-dibutyryl-2-linoleoylglycerol, 1,3-dibutyryl-2-stearoylglycerol, 1-butyryl-2-oleoyl-3-palmitoylglycerol, 1-palmitoyl-2-oleoyl-3-butyrylglycerol, 1-butyryl-2-oleoyl-3-linoleoylglycerol, 1-linoleoyl-2-oleoyl-3-butyrylglycerol, 1-oleoyl-2-butyryl-3-linoleoylglycerol, 1-linoleoyl-2-butyryl-3-oleoylglycerol, 1-butyryl-2-linoleoyl-3-oleoylglycerol, 1-oleoyl-2-linoleoyl-3-butyrylglycerol, 1-butyryl-2-stearoyl-3-oleoylglycerol, 1-oleoyl-2-stearoyl-3-butyrylglycerol, 1-butyryl-2-oleoyl-3-stearoylglycerol, 1-stearoyl-2-oleoyl-3-butyrylglycerol, 1,2-dioleoyl-3-palmitoylglycerol, 1-palmitoyl-2,3-dioleoylglycerol, 1,2-dioleoyl-3-linoleoylglycerol and/or 1-linoleoyl-2,3-dioleoylglycerol.

In one embodiment tributyrin comprises less than 10% by weight of the total butyrate moiety containing triglycerides in the composition, preferably less than 8% by weight, more preferably less than 5% by weight of the total butyrate moiety containing triglycerides in the composition.

The composition of the present invention can be in, for example, a solid (e.g. powder), liquid or gelatinous form.

The composition of the present invention can be in, for example, tablet, dragee, capsule, gel cap, powder, granule, solution, emulsion, suspension, coated particle, spray-dried particle or pill.

The composition may in the form of a pharmaceutical composition and may comprise one or more suitable pharmaceutically acceptable carriers, diluents and/or excipients. Examples of such suitable excipients for compositions described herein may be found in the “Handbook of Pharmaceutical Excipients”, 2nd Edition, (1994), Edited by A Wade and P J Weller. Acceptable carriers or diluents for therapeutic use are also well known in the pharmaceutical art, and are described, for example, in Remington's Pharmaceutical Sciences, Mack Publishing Co. (A. R. Gennaro edit. 1985).

The pharmaceutical compositions may comprise as, or in addition to, the carrier, excipient or diluent any suitable binder(s), lubricant(s), suspending agent(s), coating agent(s) and/or solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like.

Preservatives, stabilisers, dyes and even flavouring agents may be provided in the composition. Examples of preservatives include sodium benzoate, sorbic acid and esters of p-hydroxybenzoic acid. Antioxidants and suspending agents may be also used.

The composition may be a nutritional composition.

The expression “nutritional composition” means a composition that nourishes a subject. This nutritional composition is preferably taken orally, and it may include a lipid or fat source and a protein source. It may also contain a carbohydrate source. In one embodiment, the nutritional composition contains only a lipid or fat source. In other specific embodiments, the nutritional composition contains a lipid (or fat) source with a protein source, a carbohydrate source or both.

In some specific embodiments, the nutritional composition according to the invention is an “enteral nutritional composition” that is to say a foodstuff that involves the gastrointestinal tract for its administration. The gastric introduction may involve the use of a tube through the oro/nasal passage or a tube in the belly leading directly to the stomach. This may be used especially in hospitals or clinics.

In some specific embodiments, the composition is an oral nutritional supplement (ONS), a complete nutritional formula, a pharmaceutical, a medical or a food product. In some specific embodiments, the composition is administered to the individual as a beverage. The composition may be stored in a sachet as a powder and then suspended in a liquid such as water for use.

The composition according to the invention can be a dietary supplement.

The term “dietary supplement” may be used to complement the nutrition of an individual (it is typically used as such but it might also be added to any kind of compositions intended to be ingested). It may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The dietary supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

When the composition is a supplement, it can be provided in the form of unit doses.

The composition according to the invention can be a dairy product, a liquid beverage, a beverage powder, a dehydrated soup, a dietary supplement, a meal replacement, a nutritional bar, a cereal, a confectionery product or a dry pet food.

The composition may further comprise dietary fiber. The “dietary fiber” may comprise at least one non-digestible oligosaccharide (e.g. prebiotics). The prebiotics may be present in an amount between 0.3 and 10% by weight of composition. Dietary fiber and/or prebiotics may promote the production of endogenous butyrate by gut microflora and thus provide additional beneficial effects.

Prebiotics are usually non-digestible in the sense that they are not broken down and absorbed in the stomach or small intestine and thus remain intact when they pass into the colon where they are selectively fermented by the beneficial bacteria. Examples of prebiotics include certain oligosaccharides, such as fructooligosaccharides (FOS), inulin, xylooligosaccharides (XOS), polydextrose or any mixture thereof. In a particular embodiment, the prebiotics may be fructooligosaccharides and/or inulin. In a specific embodiment, the prebiotics is a combination of FOS with inulin such as in the product sold by BEN EO-Orafti under the trademark Orafti® oligofructose (previously Raftilose®) or in the product sold by BENEO-Orafti under the trademark Orafti® inulin (previously Raftiline®). Another example is a combination of 70% short chain fructooligosaccharides and 30% inulin, which is registered by Nestle under the trademark “Prebio 1”. The nutritional composition of the invention can also comprise at least one milk's oligosaccharide that can be a BMO (bovine milk oligosaccharide) and/or a HMO (human milk oligosaccharide).

The composition of the present invention can further comprise at least one probiotic (or probiotic strain), such as a probiotic bacterial strain. Consumption of probiotic strains may also promote the production of endogenous butyrate by gut microflora and thus provide additional beneficial effects.

The probiotic microorganisms most commonly used are principally bacteria and yeasts of the following genera: Lactobacillus spp., Streptococcus spp., Enterococcus spp., Bifidobacterium spp. and Saccharomyces spp.

In some particular embodiments, the probiotic is a probiotic bacterial strain. In some specific embodiments, it is Bifidobacteria and/or Lactobacilli.

The nutritional composition according to the invention may contain from 10e3 to 10e12 cfu of probiotic strain, more preferably between 10e7 and 10e12 cfu such as between 10e8 and 10e10 cfu of probiotic strain per g of composition on a dry weight basis.

In one embodiment the probiotics are viable. In another embodiment the probiotics are non-replicating or inactivated. It may also be probiotic parts such as cell wall components or products of the probiotic metabolism. There may be both viable probiotics and inactivated probiotics in some other embodiments. The nutritional composition of the invention can further comprise at least one phage (bacteriophage) or a mixture of phages, preferably directed against pathogenic Streptococci, Haemophilus, Moraxella and Staphylococci.

The nutritional composition of the invention, generally contains a protein source, a carbohydrate source and a lipid source. In some embodiments however, especially if the nutritional composition of the invention is a supplement or a fortifier, there may be only lipids (or a lipid source).

The nutritional composition according to the invention may contain a protein source. Protein sources based on, for example, whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions. In some embodiments the protein source is whey predominant (i.e. more than 50% of proteins are coming from whey proteins, such as 60%>or 70%>). The proteins may be intact or hydrolysed or a mixture of intact and hydrolysed proteins. In some embodiments, the protein source may also be provided partially or entirely in the form of added amino acids.

By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein, which has been hydrolysed or broken down into its component amino acids.

The proteins may be either fully or partially hydrolysed. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10%>by weight of lysine; for example about 7% by weight of lysine which greatly improves the nutritional quality of the protein source.

In one particular embodiment the proteins of the composition are hydrolysed, extensively hydrolysed or partially hydrolysed. The degree of hydrolysis (DH) of the protein can be between 2 and 20, or between 8 and 40, or between 20 and 60 or between 20 and 80 or more than 10, 20, 40, 60, 80 or 90. For example, nutritional compositions containing hydrolysates having an extent of hydrolysis less than about 15% are commercially available from Nestle Company under the trade mark Peptamen®.

In some embodiments the protein is extensively hydrolysed.

At least 70%, 80%, 85%, 90%, 95% or 97% of the proteins may be hydrolysed. In a particular embodiment, 100% of the proteins are hydrolysed.

In one particular embodiment the proteins are provided as amino acids.

In one particular embodiment the proteins of the composition are plant based protein.

The nutritional composition according to the present invention may contain a carbohydrate source. Any carbohydrate source conventionally found in nutritional compositions such as lactose, sucrose, saccharose, maltodextrin, starch and mixtures thereof may be used.

The nutritional composition of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B3, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

If necessary, the nutritional composition of the invention may contain emulsifiers and stabilisers such as soy, lecithin, citric acid esters of mono- and diglycerides, and the like. The nutritional composition of the invention may also contain other substances which may have a beneficial effect such as lactoferrin, osteopontin, TGFbeta, sIgA, glutamine, nucleotides, nucleosides, and the like.

The nutritional composition according to the invention may be prepared in any suitable manner. For example, a composition may be prepared by blending together the components in appropriate portions, optionally blended with one or more carriers and then mixing the dry blended mixture with a liquefier to form a liquid mixture. The liquid mixture may then be homogenised, pasteurised and optionally spray-dried if the final product is to be a powder. The composition may be homogenised before pasteurisation or after pasteurisation.

The nutritional composition of the invention can be administered to an individual such as a human, e.g., an elderly human, in a therapeutically effective dose. The therapeutically effective dose can be determined by the person skilled in the art and will depend on a number of factors known to those of skill in the art, such as the severity of the condition and the weight and general state of the individual.

In one embodiment of the invention, the nutrition composition is administered to a subject in combination with a regime of exercise or physical activity.

The nutritional composition of the invention can be formulated to be administered to an animal, in the form of animal treats (e.g., biscuits), or dietary supplements. The compositions may be a dry composition (e.g., kibble), semi-moist composition, wet composition, or any mixture thereof. In another embodiment, the nutritional composition is a dietary supplement such as a gravy, drinking water, beverage, yogurt, powder, granule, paste, suspension, chew, morsel, treat, snack, pellet, pill, capsule, tablet, or any other suitable delivery form.

The nutritional composition may be administered to an individual in an amount sufficient to prevent or at least partially reduce the risk of developing a cardiovascular disorder where a cardiovascular disorder has yet not been developed in the individual. Such an amount is defined to be “a prophylactically effective dose.”

The nutritional composition is preferably administered as a supplement to the diet of an individual daily or at least twice a week. In an embodiment, the composition is administered to the individual consecutively for a number of days. For example, the composition can be administered to the individual daily for at least 30, 60 or 90 consecutive days. As another example, the composition can be administered to the individual for a longer period, such as a period of 1, 2, 3, 4, 5, 6, 7, 8, 9 or 10 years.

In one preferred embodiment, the nutritional composition is administered to the individual for at least 3 months, for example a period of 3 months to 1 year, and preferably for at least 6 months.

The above examples of administration do not require continuous daily administration with no interruptions. Instead, there may be some short breaks in the administration, such as a break of two to four days during the period of administration. The ideal duration of the administration of the composition can be determined by those of skill in the art.

### Cardiovascular Disorders

The invention provides compounds, compositions and methods of preventing and/or treating cardiovascular disorders by reducing residual inflammatory risk.

Cardiovascular disorders affect the heart or blood vessels. A major cardiovascular disorder is atherosclerosis (also known as arteriosclerotic vascular disease) which is a condition in which patchy deposits of fatty material (atheromas or atherosclerotic plaques) develop in the walls of medium-sized and large arteries, leading to reduced or blocked blood flow. Atherosclerosis is one form of arteriosclerosis, which means hardening of the arteries. Arteriosclerosis interferes with the body's control of blood pressure, increasing the risk of high blood pressure. The stiffness of the arteries prevents the dilation that would otherwise return blood pressure to normal. People with high blood pressure are at a greater risk of stroke, heart attack, and kidney failure.

Atherosclerosis is a syndrome affecting arterial blood vessels, a chronic inflammatory response in the walls of arteries, caused largely by the accumulation of macrophages and promoted by low-density lipoproteins (LDL, plasma lipoproteins that carry cholesterol and triglycerides) without adequate removal of fats and cholesterol from the macrophages by functional high density lipoproteins (HDL). It is commonly referred to as a hardening or furring of the arteries. This is caused by the formation of multiple plaques within the arteries.

Where atherosclerosis affects the arteries supplying blood to the heart (coronary artery disease) this can cause chest pain (angina pectoris) or a heart attack where an area of heart muscle is destroyed (myocardial infarction). The reduction of the flow of oxygen-rich blood to the heart muscle can cause heart failure, a disorder in which the heart pumps blood inadequately, leading to reduced blood flow, back-up (congestion) of blood in the veins and lungs, and other changes that may further weaken the heart. The failure of coronary circulation to supply adequate circulation to cardiac muscle and surrounding tissue is called coronary heart disease.

Atherosclerosis that affects the arteries to the brain leads to strokes. A stroke occurs when an artery to the brain becomes blocked or ruptures, resulting in death of an area of brain tissue (cerebral infarction).

Although the major cardiovascular disorders in terms of mortality are strokes and heart attacks, cardiovascular disorders also encompass such conditions as aortic aneurysms and peripheral vascular disease and contribute to clinical conditions including renal vascular disease, vascular dementia and retinal disease. Preventing cardiovascular disorders is not just about reducing mortality, but also about preventing disability and improving quality of life.

It may be appreciated that the compounds, compositions and methods of the present invention may be beneficial to prevent and/or treat atherosclerosis and/or related conditions, in particular, to prevent atherosclerosis initiation or progression.

### Method of Treatment

It is to be appreciated that all references herein to treatment include curative, palliative and prophylactic treatment; although in the context of the invention references to preventing are more commonly associated with prophylactic treatment. Treatment may also include arresting progression in the severity of a disease.

The term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder. As used herein, a subject is “in need of a treatment if such subject would benefit biologically, medically or in quality of life from such treatment.

### Subject

The term “subject” means any animal, including humans and companion animals. Generally, the subject is a human or an avian, bovine, canine, equine, feline, hircine, murine, ovine or porcine animal. The subject can be a horse or a companion animal, for example a cat or a dog. Preferably, the subject is a human.

The treatment of mammals, particularly humans, is preferred. However, both human and veterinary treatments are within the scope of the invention.

For veterinary subjects, dogs, cats and equine subjects are preferred.

### Administration

Preferably, the compounds and compositions described herein are administered enterally.

Enteral administration may be oral, gastric, and/or rectal.

In one embodiment the administration is oral or gastric. In a preferred embodiment administration is oral.

In general terms, administration of the combination or composition described herein may, for example, be by an oral route or another route into the gastro-intestinal tract, for example the administration may be by tube feeding.

The subject may be a mammal such as a human, canine, feline, equine, caprine, bovine, ovine, porcine, cervine and primates. Preferably the subject is a human.

### Organoleptic Properties

The present invention provides compounds that are a source of butyrate having improved organoleptic properties. In particular, the compounds have improved odor and/or taste relative to butyric acid, butyrate salts and/or tributyrin. In one embodiment, the compounds have improved taste relative to tributyrin. In one embodiment, the compounds have improved smell relative to butyrate salts (e.g. sodium butyrate).

In one embodiment, the improved organoleptic properties are improved odour. In one embodiment, the improved organoleptic properties are improved taste. In one embodiment, the improved organoleptic properties are improved odour and improved taste. In one embodiment, the improved taste is reduced bitterness.

### EXAMPLES

### Example 1—Preparation of Butyrated Triglycerides (TAG)

Compositions comprising butyrated TAG were generated by chemical interesterification between tributyrin and high oleic sunflower oil in the presence of catalyst such as sodium methoxyde. A molar excess of tributyrin compared to high oleic sunflower oil was be used.

The three reagents, tributyrin, high oleic sunflower oil and the catalyst were mixed together into a reactor under nitrogen atmosphere and then heat under stirring at 80° C. for 3h. Once the reaction is completed, the product was washed several times with water then dried under vacuum (25 mBar at 60° C. for 2h). The resulting oil product was then subjected to a decoloration step with the action of bleaching earth and was purified either by short-path distillation (130° C., 0.001-0.003 mbar) or by deodorisation (160° C., 2 mbar, 2h) with injection of steam water.

The constituents, mostly triglycerides, of the resulting oil compositions are shown below in Table 1. These triglycerides are represented by the three fatty acids they contain. These fatty acids are represented by their lipid number: 4:0 for butyrate, 16:0 for palmitate, 18:0 for stearate, 18:1 for oleate and 18:2 for linoleate. The fatty acid in the middle is located on the position sn-2 in the triglyceride. As an example, 16:0-4:0-18:1 stands for two different triglycerides having both a butyrate in position sn-2 and either a palmitate in position sn-1 and an oleate in position sn-3 or an oleate in position sn-1 and a palmitate in position sn-3.

TAG profile and regioisomers were analyzed by liquid chromatography coupled to high resolution mass spectrometer. Lipid classes' proportion was evaluated by liquid chromatography coupled to evaporative light scattering detector (ELSD).

TABLE 1
TAG regioisomer profile [g/100 g]
TAG regioisomer [g/100 g]
Composition

| | |
|---|---|
| 4:0-4:0-4:0 | <0.4-4.7 |
| 4:0-16:0-4:0 | 0.8-1.0 |
| 4:0-18:2-4:0 | 4.0-6.3 |
| 4:0-4:0-18:1 | 3.0-6.1 |
| 4:0-18:1-4:0 | 16.2-27.0 |
| 4:0-18:0-4:0 | 0.8-1.3 |
| 4:0-22:0-4:0 | ≤0.4 |
| 4:0-16:0-18:1 | 1.1-1.5 |
| 16:0-4:0-18:1 | 0.5-0.7 |
| 4:0-18:1-16:0 | 1.2-1.6 |
| 4:0-18:1-18:2 | 2.6-3.1 |
| 18:1-4:0-18:2 | 1.1-1.6 |
| 4:0-18:2-18:1 | 2.9-3.6 |
| 18:1-18:1-4:0 | 23.3-25.8 |
| 18:1-4:0-18:1 | 3.3-4.8 |
| 4:0-18:0-18:1 | 0.9-1.3 |
| 4:0-18:1-18:0 | 0.8-1.1 |
| 4:0-22:0-18:1 | <0.4-0.5 |
| 18:1-18:1-16:0 | 0.6-1.4 |
| 18:1-18:1-18:2 | 1.3-1.5 |
| 18:1-18:2-18:1 | 0.5-0.7 |
| 18:1-18:1-18:1 | 6.1-10.7 |
| 18:1-18:1-18:0 | 0.5-0.8 |
| Total | 93.1-94.1 |

In the Composition samples, the two most abundant TAG are 4:0-18:1-4:0 and 18:1-18:1-4:0, they represent together approximately 40 to 50 g/100 g.

### Example 2—Odor Properties of Butyrate Moiety Containing Triglycerides

An odor comparison of a solution including butyrate moiety containing TAG (composed mainly with oleic and butyric fatty acids) was compared to a solution containing sodium butryate.

### Sample Preparation

Solutions including butyrate moiety containing TAG (see Example 1) or sodium butyrate were prepared and stored at 4° C. prior to delivery to the sensory panel. Each 250 mL solution contained 600 mg of butyric acid (equivalent to one capsule of commercially available sodium butyrate as a supplement; 2.4 mg/mL concentration) and 1% w/v BEBA Optipro 1 infant formula in acidified, deionized water.

The samples were prepared the day before the test, by putting 4 mL of each solution (TAG butyrate solution; sodium butyrate solution) in Agilent vials.

### Methodology

The ‘two-out-of-five test’ was performed. In this test, the panellist is given five samples. The panellist is instructed to identify the two samples that are different from the other three. The presentation order of the samples is randomized in order to avoid presentation order bias.

In addition to the two-out-of-five test, a comment box was presented to the panellists to allow them to comment about the nature of the difference perceived (e.g. odour intensity, odour quality).

### Results

The five samples were presented simultaneously to the panellists. They were asked to uncap, smell and then cap each vial in a given order. The results are shown in Table 2.

TABLE 2

| Number of | Number of correct | |
|---|---|---|
| responses | responses | Significance |
| 11 | 9 | p < 0.0001*** |

P-value was calculated using a binomial test performed with Fizz software (Biosystemes, France).

The panellists who found the correct responses (butyrate moiety containing TAG different from sodium butyrate) mentioned that the sodium butyrate smells “cheese” whereas for the butyrate moiety containing TAG samples this “cheese” smell was considerably decreased and the odour was quite neutral.

### Example 3—Taste Properties of Butyrate Moiety Containing Triglycerides

Sensory benchmarking of a solution including butyrate moiety containing TAG (see Example 1) composed mainly with oleic and butyric fatty acids was performed versus a solution containing tributyrin.

### Sample Preparation:

One scoop (4.6g) of BEBA Optipro 1 infant formula was added to warm water (cooled, boiled tap water as per instructions) to a final volume of 150 mL (approximately 3% w/v solution). Each TAG form of butyrate was weighed separately to deliver 600 mg of butyrate, and the addition of infant formula to a final volume of 50 mL for each solution was performed.

Solution A included butyrate moiety containing TAG (see Example 1); and solution B contained tributyrin.

### Methodology

A group of panellists performed a repeated blind-coded tasting.

The samples were prepared just prior to the preliminary bitterness assessment, and each solution was vigorously shaken. Tasting cups labelled A and B were filled at the same time with a small volume of the respective solution.

The two samples were presented simultaneously to the panellists. They were asked to taste the solution in a sip and spit fashion, and rank the perceived bitterness on a scale from 0-10; where 0 is no bitterness perceived and 10 resembles the maximum imaginable bitterness.

### Results

Bitterness of Solution A was ranked by panellists at 4.33±1.52, mean±SD.

Bitterness of Solution B was ranked by panellists at 8.33±1.52, mean±SD.

These data show that the butyrate moiety containing TAG composition in infant formula was notably less bitter in taste as compared to tributyrin in infant formula.

### Example 4—Taste Properties 1,3-Dibutyryl-2-Palmitoylglycerol

1,3-dibutyryl-2-palmitoylglycerol (BPB) was synthesized as a single compound using the following synthesis:

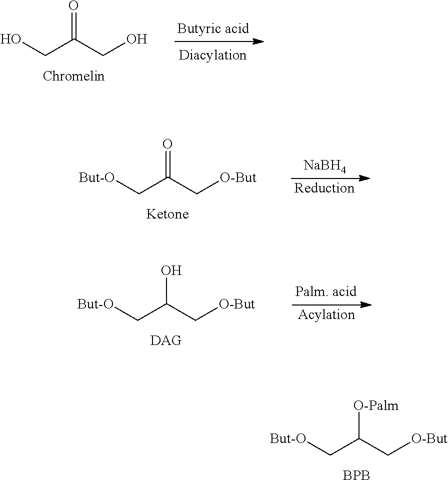

BPB was evaluated in a descriptive sensory panel evaluation and found to be neutral in taste and odor.

### Example 5—Digestion of Butyrate Moiety Containing Triglycerides

### 5.1 Emulsion Preparation

10 wt % oil in water emulsions stabilised by 0.3 wt % polyoxyethylene sorbitan mono-oleate (Tween® 80) were prepared by mixing the Tween 80 into the oil phase at 40° C., then mixing with the water phase. An emulsion was then created using an ultrasonic probe homogeniser.

### 5.2 Granulometry

The droplet size of each lipid emulsion was measured by laser light scattering using a Mastersizer 3000 equipped with a Hydro SM from Malvern Instruments (Malvern, Worcestershire, United Kingdom). Emulsion particle sizes are quoted as two values, the volume surface mean diameter D3,2 (D3,2 ¼ Pnidi 3/nidi 2) or the volume length mean diameter D4,3 (D4,3 ¼ Pnidi 4/nidi 3). Emulsion particle size results are an average of three measurements of two freshly prepared emulsions.

### 5.3 In Vitro Digestion

The lipid emulsion (2 mL) containing 200 mg of fat was subjected to gastrointestinal in vitro lipolysis. The digestions were conducted in thermostated glass vessels (37° C.) in a pH-STAT setup controlled by a TIM 856 bi-burette pH-STAT (Radiometer Analytical, France). For gastric digestion, the sample was incubated for 90 minutes with 8.5 mL of simulated gastric fluid (SGF), which consisted of 150 mM NaCl, 450 U/mL pepsin, 18 U/mL rabbit gastric lipase at 37° C. and a pH of 5.5. The digestion was initiated by adding 18 tributyrin U/ml (TBU) activity determined at pH 5.4) of rabbit gastric lipase.

The intestinal digestion step was performed in the pH stat where the pH was kept constant at 6.8 by addition of NaOH. A bile salt mixture (bile salts prepared with tris buffer) and calcium solution (20 mM Ca, 176 5 mM tris, 150 mM NaCl) were added to the SGF-sample mixture. This mixture was transferred to the pH-stat, where the pH was adjusted to approximately 6.78. The intestinal digestion step starts when the temperature reaches 37±0.5 ° C. The pH was adjusted to pH 6.8 and after incubation of two minutes at this pH and temperature, a pancreatin solution was added. The final composition of the intestinal fluid was 10 mM CaCl₂, 12 mM mixed bile salts, 0.75 mM phospholipid, 150 mM NaCl and 4 mM tris(hydroxymethyl)aminomethane buffer. The intestinal digestion step was carried out for 3 hours in a titration manager from Radiometer. During the intestinal phase of digestion, the kinetics of digestion were followed using a pH-stat (TIM856, Radiometer) technique and expressed as titratable acid (rather than fatty acid) that was calculated by the equation:

TA=V_{NaOH}×0:05×1000

TA: Total titratable acid released, mmol; V_{NaoH}: volume of NaOH used to titrate the released acid in 3 h, mL.

### 5.6 Results

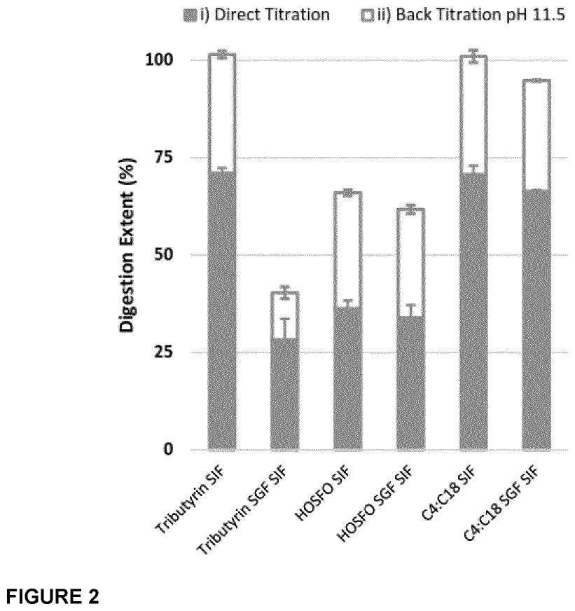

Since the digestion of dietary lipids involves lipases of both gastric and intestinal origin, lipid digestibility was assessed using two digestion models i) simulated intestinal fluid (SIF) with porcine pancreatic lipase (PPL) and ii) sequential digestion in simulated gastric fluid (SGF) with rabbit gastric lipase (RGL) followed by simulated intestinal fluid (SIF) with porcine pancreatic lipase (PPL). All lipids were emulsified using polyoxyethylene sorbitan mono-oleate (Tween® 80) and had similar particle size distributions and specific surface areas (FIG. 2), meaning the differences in digestion are predominately arising from the triglyceride molecular structure.

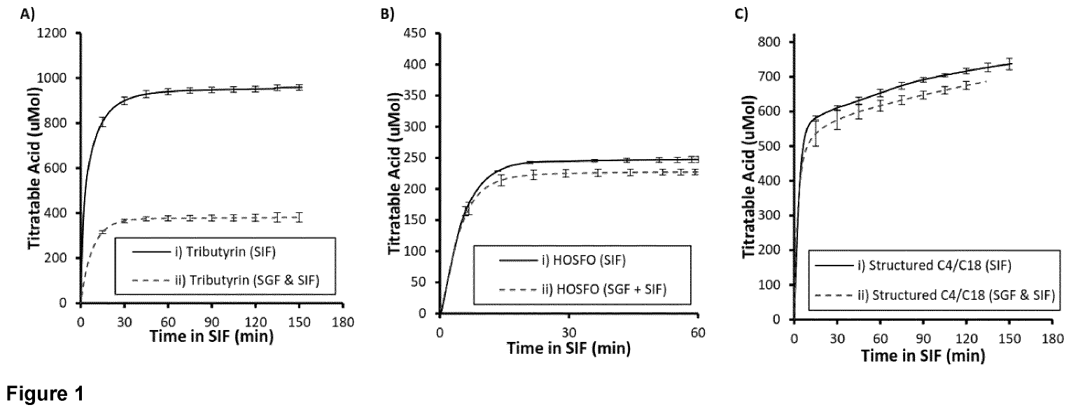

FIG. 1i A-C shows the digestion of tributyrin (C4), high oleic sunflower oil (HOSFO, largely C18:1) and butyrate moiety containing triglycerides according to the invention, generated by chemical interesterification between tributyrin and high oleic sunflower oil (see Example 1) “C4-C18:1”, by porcine pancreatic lipase (from pancreatin) in the presence of mixed bile and calcium (SIF model). The lipids generally exhibit the same lipolysis behaviour, undergoing an initial rapid period of lipolysis during the first 15 minutes which progressively slows during the final 2.5 hours of simulated intestinal digestion. C4 triglyceride exhibited an initial maximal rates of lipolysis of 223±59 μmol.min⁻¹. The initial rate of lipolysis for the high oleic sunflower oil, 34.5±2.3 μmol.min⁻¹ was significantly lower (p<0.0001) than the short chain triglyceride. C4-C18:1 exhibited an initial rate of hydrolysis of 153±47 μmol.min⁻¹, between that of the C4 and C18:1. Overall, it is seen that all of the triglycerides are rapidly and extensively digested in the presence of porcine pancreatic lipase.

The triglycerides were next digested using the sequential SGF (RGL) SIF (PPL) model, the digestion in the SIF compartment is shown in FIG. 1ii A-C. No measurements were taken in the gastric compartment due to limited ionisation of the target fatty acids. Compared to when they were digested with SIF alone, the C4 and C18:1 triglycerides generally released a lower amount of titratable acid during 3 hours of digestion. The effect is largest with tributyrin, which has a significantly lower (p<0.0001) initial lipolysis rate 44.1±8.8 μmol.min⁻¹ during SGF-SIF digestion compared to SIF alone 223±59 μmol.min⁻¹. The total amount of acid released after SGF-SIF digestion of tributyrin 381±20 μmol, is almost ⅓ the amount released after SIF only digestion, 958±12.5 μmol. These results clearly indicate that there is considerable digestion of tributyrin within the gastric compartment of the model.

When sequentially exposed to SGF and SIF, the SIF lipolysis rates of the butyrate moiety containing triglycerides C4-C18:1 is 124±20 μmol.min⁻¹, showing a slight but not significant decrease compared to SIF alone (124±20 μmol.min⁻¹). The most interesting observation is the influence of secondary fatty acid chain length on the decrease in SIF lipolysis caused by RGL pre-exposure. Originally, tributyrin exhibited a 60.2% (147±7.6 μmol) decrease in total fatty acid release during SIF lipolysis after pre-exposure to RGL in SGF. In comparison, the C4-C18:1 interesterified triglycerides exhibited a 6.1% (45±7.6 μmol) decrease.

The overall extent of lipid digestion after both SIF and SGF-SIF is presented in FIG. 2 for the three triglycerides using direct and back titration. Because many fatty acids are only partially ionised at pH 6.8, direct titration gives only partial picture of the extent of lipid digestion, instead back titration to pH 11.5 or GC-FAME analysis is required to estimate the full extent of digestion. Results of the back titration for the three triglycerides show that tributyrin and the butyrate moiety containing triglycerides C4-C18:1 underwent 101.5±0.9% and 101±1.6% digestion respectively, indicating release of three fatty acids per molecule for complete digestion, whilst high oleic sunflower oil underwent 72.3±2% digestion indicating release of two fatty acids per molecule for complete digestion.

Overall, it was seen that tributyrin underwent extensive hydrolysis in the stomach, whilst high oleic sunflower oil triglyceride underwent very limited hydrolysis in the stomach. Surprisingly, it was seen that butyrate moiety containing triglycerides generated via interesterification of C4 with long chain fatty acids (C4-C18:1) decreases the extent of gastric lipolysis of C4 fatty acids. Tributyrin underwent ˜60% lipolysis by gastric lipase as indicated by decreased total fatty acid release during SIF lipolysis after pre-exposure to RGL in SGF. In comparison, the C4-C18:1 butyrate moiety containing triglycerides exhibited only a 6.1% decrease in total fatty acid release in SGF-SIF. These results suggest that interesterification of C4 with long chain fatty acids (C4-C18:1) modulates the release of butyric acid within the stomach to later in the intestine following digestion, and that the design of structured lipids alter the timing (but not extent) of short chain fatty acid delivery in the gastrointestinal tract.

All publications mentioned in the above specification are herein incorporated by reference. Various modifications and variations of the disclosed methods, cells, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for the prevention or treatment of cardiovascular disorders comprising administering to an individual in need of same a compound having the formula 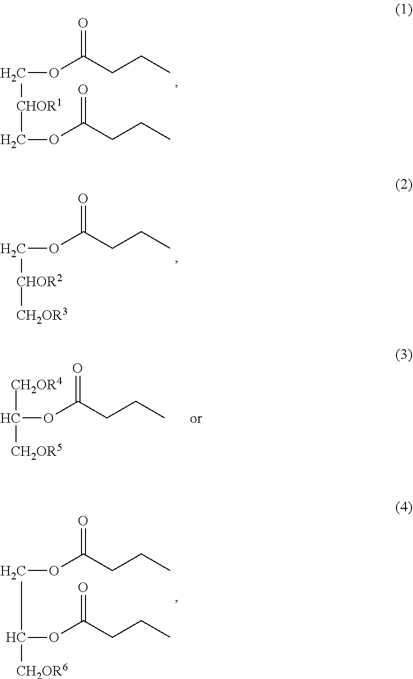
or combinations thereof, wherein R¹, R², R³, R⁴, R⁵ and R⁶ are independently, a long chain fatty acid having between 16 and 20 carbons.

2. A method for the prevention or treatment of cardiovascular disorders comprising administering to an individual in need of same a composition comprising a compound having the formula 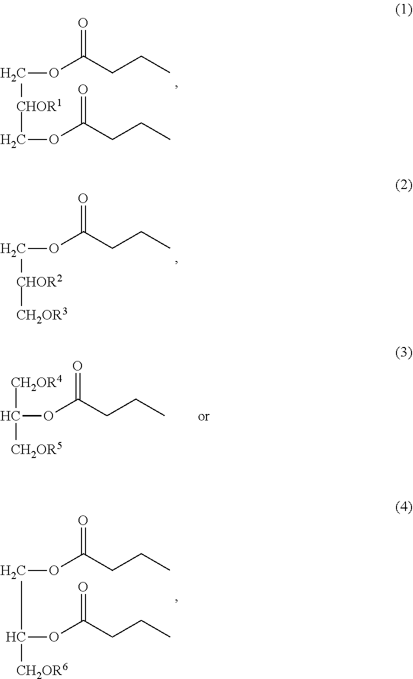
or combinations thereof, wherein R¹, R², R³, R⁴, R⁵ and R⁶ are independently, a long chain fatty acid having between 16 and 20 carbons.

3. A method according to claim 2, wherein the composition comprises the compound having formula (1), the compound having formula (2), the compound having formula (3) and the compound having formula (4).

4. A method according to claim 2, wherein the compounds having formula (1), (2), (3) and (4), comprise at least 50% by weight of the total triglycerides of the composition.

5. A method according to claim 2, wherein the compounds having formula (1), (2), (3) and (4), comprise at least 50% by weight of the total butyrate moiety containing triglycerides in the composition.

6. A method according to claim 2, wherein tributyrin comprises less than 10% by weight of the total triglycerides in the composition.

7. A method according to claim 2, wherein the composition further comprises dietary fiber.

8. A method according to claim 2, wherein the composition is in a form selected from the group consisting of a nutritional composition, a dietary supplement and a pet care product.

9. A method according to claim 1, wherein R¹, R², R³, R⁴, R⁵ and/or R⁶ is an unsaturated fatty acid, preferably monounsaturated.

10. A method according to claim 1, wherein R¹, R², R³, R⁴, R⁵ and/or R⁶ is selected from the group consisting of oleic acid, palmitic acid, and linoleic acid.

11. A method according to claim 1, for the treatment of prevention of atherosclerosis, coronary heart disease, myocardial infarction, angina pectoris, stroke, and heart failure.

12. A method according to claim 2, for use in the prevention of atherosclerosis.

13. A method of treatment or prevention of cardiovascular disorders, comprising administering to a human or animal subject an effective amount of a compound having the formula 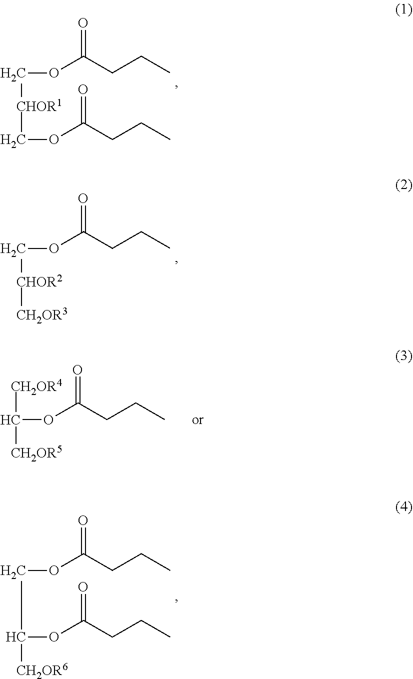
or combinations thereof, wherein R¹, R², R³, R⁴, R⁵ and R⁶ are independently, a long chain fatty acid having between 16 and 20 carbons.

14. A method of treatment or prevention of cardiovascular disorders according to claim 13, wherein the cardiovascular disorder is selected from the group consisting of atherosclerosis, coronary heart disease, myocardial infarction, angina pectoris, stroke, and/or heart failure.

15. A method according to claim 2, or a method according to claim 13 or 1 d claim 2, wherein the compounds or combinations thereof have improved organoleptic properties relative to butyric acid, tributyrin and/or butyrate salts.
